(19) United States
(12) Patent Application Publication
(10) Pub. No.: US 2014/0212502 A1
(43) Pub. Date: Jul. 31, 2014

(54) NANO- AND MICRO-BUBBLES WITH ULTRASOUND-TRIGGERED RELEASE AND IMAGING FUNCTIONALITIES

(71) Applicant: NATIONAL YANG-MING UNIVERSITY, Taipei City (TW)

(72) Inventor: Tse-Ying LIU, TAIPEI CITY (TW)

(73) Assignee: NATIONAL YANG-MING UNIVERSITY, Taipei City (TW)

(21) Appl. No.: 13/955,987

(22) Filed: Jul. 31, 2013

(30) Foreign Application Priority Data
Jan. 31, 2013 (TW) 102103694

(51) Int. Cl. A61K 9/00

(52) U.S. Cl. CPC A61K 9/0009
USPC 424/493; 424/490; 514/44 R

(57) ABSTRACT

The present invention relates to a novel nano-/micro-bubble drug vehicle with functions of carrying hydrophobic drugs, ultrasound-triggered release and magnetic resonance or optical imaging, made of amphiphilic chitosan polymer material, and lipophlic superparamagnetic iron oxide (SPIO) or luminous nanoparticles. By using magnetic resonance or optical imaging to track the location of the drug vehicle, a user can trigger the release of drug by ultrasonication when the drug vehicle arrives at target site and accumulates to a desirable concentration. The nano-/micro-bubble drug delivery system provides improved accuracy of drug releasing, including position and timing, and thus reduces side effects of the drug. In addition, the synergistic effect of the amphiphilic chitosan molecules and sonication may improve the transmembrane delivery of hydrophobic agent into target cell, and enhance the cytotoxicity of anti-cancer drugs.

### FIELD OF THE INVENTION

The present invention relates to a nano- or micro-bubble with functions of encapsulating hydrophobic agents, ultrasound (US)-triggered release and magnetic resonance (MR) imaging or optical imaging. Especially, the present invention relates to a multi-functional drug delivery bubble made of amphiphilic chitosan, and lipophilic superparamagnetic iron oxide (SPIO) or lipophilic luminous nano-particles or lipophilic quantum dot.

### BACKGROUND OF THE INVENTION

Current ultrasound image-guided drug vehicles mainly use microbubble to carry drugs. Microbubble is a widely used commercial ultrasound contrast agent, for which the ultrasound contrast agent may performance the function of trafficking and ultrasonically-triggered drug releasing.

However, the ultrasonically-triggered drug delivery systems might encounter following issues: (1) “seeing” the contrast agent at non-affected area in a series of image probing, the drug may also be released by the triggering at non-affected area, which makes the mentioned drug delivery method more difficult to control the timing and location of release; (3) Microbubbles (MBs) are common contrast agent used in US-image-guided drug delivery systems. MBs cannot be employed as an agent for extravascular imaging and delivery owing to the fact that micronized objects circulating in blood capillaries cannot easily pass through the leaky vasculature (having fenestrations sizes of 100 nm to 1.5 μm depending on the tumor type) in tumor tissue to access the blood capillary-surrounding tumor cells (i.e., extravasation effect); and (3) the US echo signals of nanobubble are very weak, and the attenuation of US imaging signals is hard to provide a molecular image with high resolution in an animal body by ordinary clinical US image scanner.

Based on the above considerations, a nano- and micro-bubble with MR imaging function is provided in this invention to be used as an image-guided drug vehicle, in which nano-bubbles can provide extravascular imaging and US triggered release of drug, and micro-bubbles are capable of intravascular imaging, US imaging and US triggered release of drug. Moreover, MR imaging and US imaging cannot provide vehicle image in target cell, so loading of lipophilic zinc oxide or quantum dot nanoparticles on the drug vehicle of this invention will confer the vehicle with optical imaging.

Albumin- and lipid-based micro-bubbles are commonly used in conventional US image-guided drug vehicle as a contrast agent and drug vehicle (Duvshani-Eshet, M. et. Al., Journal of Controlled Release 112, 156-166 (2006); Vlaskou D et. Al., Advanced Functional Materials 20, 3881-94 (2010)). It is reported that such micro-bubbles also exhibit MR imaging effect, but their MR imaging effect is limited by lacking magnetic materials, and the optical imaging effect is insufficient and hard to provide a sharp image for clinic diagnostic when the concentration is too low.

The drugs carried in conventional US image-guided drug vehicles using albumin- and lipid-based micro-bubbles are commonly contained in oil drops encapsulated in the micro-bubbles (Tinkov, S. et. al, Journal of Controlled Release 148, 368-372 (2010)), or conjugated directly on the shell surface of micro-bubbles (Liu, Y. Y. et. al, Journal of Controlled Release 114, 89-99 (2006)). The drug carrying capacity of drug vehicles produced in these two ways is limited, and special procedures and equipment are required for preparation.

In the present invention, amphiphilic chitosan is used in the preparation of nano- and micro-bubbles. The hydrophobic anti-cancer drug is carried by the hydrophobic interaction between the hydrophobic group of amphiphilic chitosan and hydrophobic nano-particles. In embodiments of the invention, nano-bubbles carrying a hydrophobic drug are prepared by using lipophilic superparamagnetic iron oxide (SPIO) nanoparticles and amphiphilic chitosan.

In prior art of producing MR image-guided drug vehicle, a macromolecular sphere micelle is used to encapsulate drug and magnetic nano-particles (Talelli, M. et. al., Langmuir 25, 2060-2067 (2009)). However, said MR image-guided drug vehicle doesn't exhibit a function of ultrasound-triggered drug release.

Conventional multi-functional imaging drug vehicle includes a contrast agent with US- and MR-imaging functions, which is consisted of a macromolecular hollow multilayered sphere. In particular, the SF₆ (or equivalent) gas filled in the core of macromolecular hollow sphere may exhibit a function of US imaging, and the drug and magnetic particles embedding in the macromolecular shells may exhibit functions of MR imaging and drug carrier (Fang, Y. et al., Biomaterials, 30, 3882-3890 (2009)). However, the shell of such drug vehicles is too hard to be broken by US, and have no US triggered release function, in which the macromolecular material couldn't exhibit synergistic effect with sonication to improve the entrance of drugs into target cells.

On the other hand, conventional US- and MR-imaging bifunctional contrast agents may be produced by covering micro-bubbles with magnetic nano-particles in various methods (Vlaskou, D. et al., Advanced Functional Materials 20, 3881-3894 (2010); Lee, M. H. et. al., Langmuir 26, 2227-2230 (2010)); or the US- and optical-imaging bifunctional contrast agents may be produced by covering micro-bubbles with fluorescent nano-particles in various methods (Hengte Ke et. al., Nanotechnology 20, 425105 (2009)). Although these two kind of conventional bi-functional contrast agents can be broken by US, they have difficulty to carry hydrophobic drugs.

In general, it is more difficult to prepare NBs than MBs owing to their thermodynamic instability. Wang et al. used surfactant and lipid-based molecules to prepare NBs as a US contrast agent (International journal of pharmaceutics, 384, 148-153, (2010)). In addition, poly(lactic-co-glycolic acid) was employed to prepare dye-encapsulated NBs for US/optical dual-modal imaging (Xu et al., Biomaterials, 31, 1716-1722 (2010)). Xing et al. also ultrasonicated a mixture of Span 60 and polyoxyethylene 40 stearate followed by differential centrifugation to obtain NBs (in Nanotechnology 21, 145607 (2010)). Nevertheless, considerable challenges remain to be overcome to prepare multifunctional NBs exhibiting encapsulation of a hydrophobic drug, MR imaging, and US-triggered release functionalities by a facile sonication method without using surfactants. In this invention, the inhibited growth and improved hydrophobic drug carrying capacity of NBs were achieved by the hydrophobic interaction between the amphiphilic chitosan and hydrophobic nano-particles.

A related technique of preparing magnetic MBs using chemically modified magnetic nano-particles to conjugate with MBs was disclosed in Taiwan patent application no. 096105671. Problems in the chemical conjugation method of chemically modified magnetic particles were: (1) the chemical conjugation procedure will disrupt the fragile structure of MBs, and make MBs broken or eliminated; (2) the magnetic nano-particles will not selectively absorbed onto the surface of MBs but also existed in the solution environment surrounding MBs, which will interfere effects of MR imaging; (3) it is difficult for prior art to perform simultaneous loading of the magnetic nano-particles and drug in MBs.

Some researches had reported that magnetic NBs may be prepared by a fluid process (Lee, M. H. et al., Langmuir 26, 2227-2230 (2010)). However, the producing procedure and function of NB vehicles for hydrophobic drugs were not mentioned in the reports. In this invention, a novel process for preparing MBs carrying magnetic nano-particles and drug without destructing the MB's structure, and without using complicated and specified equipment. More important, superparamagnetic NBs can be prepared according to the method of this invention.

The SF₆ (or equivalent) gas filled in the NBs of this invention may exhibit a US imaging function; the bubble shell composed of amphiphilic chitosan and lipophilic imaging nanoparticles may function as a hydrophobic drug vehicle and contrast agent, which will track the image of drug delivery vehicle by MRI to confirm if adequate amount of which reach to a specific location, and trigger the drug release from delivery vehicle immediately by ultrasound to achieve the precise administration of treatment; the amphiphilic chitosan molecules dispersed after breaking of bubble shell will accelerate transmembrane delivery of hydrophobic drugs. So far, there is no similar materials and process as described in this invention ever used in preparing multifunctional NBs and MBs.

### SUMMARY OF THE INVENTION

Based on the consideration of the disadvantages mentioned above, the inventor invents to develop a multifunctional NBs and MBs exhibiting encapsulation of a hydrophobic drug, MR imaging, and US-triggered release functionalities. In the bubble delivery system of this invention, the position of drug delivery vehicle can be tracked by MR imaging to confirm if adequate amount of which reach to a target site, and the drug release from delivery vehicle is trigger by ultrasound bombardment to destroy bubble shell when the drug vehicle accumulates to a desirable concentration. By this way, the accuracy of drug releasing, including position and timing, is greatly improved, and thus reduces dosage and side effects of the drug. Additionally, synergistic effects of the amphiphilic chitosan molecules and sonication may improve the entrance of hydrophobic agent into target cell, and enhance the cytotoxicity of anti-cancer drugs.

In one aspect, the present invention provides a US-triggered drug releasing nano-/micro-bubble exhibiting encapsulation of a hydrophobic drug and imaging functionalities, which is characterized by comprising a bubble shell composed of an amphiphilic chitosan; a gaseous core filled of a water-insoluble gas; and a hydrophobic agent encapsulated in the shell incorporated with hydrophobic nanoparticles by a hydrophobic interaction. The nano-/micro-bubble of present invention may further comprise a lipophilic imaging nanoparticle to track the position of the bubble vehicle by MRI or optical imaging. The particle size of the nano-/micro-bubble of present invention may be controlled by adjusting the amount ratio of the inorganic nanoparticle, and determined to prepare a nano-bubble (with a possible particle size of 600-900 nm) or a micro-bubble (with a possible particle size of 3 μm to 10 μm).

In one embodiment of the present invention, the hydrophobic agent may be a hydrophobic anti-cancer agent. In another embodiment of the present invention, the hydrophobic agent encapsulated may be a negatively charged nucleic acid molecule. Because the prepared NBs and MBs using amphiphilic chitosan, such as CHC, as a bubble-forming material are positively charged, they can be a delivery vehicle for a negatively charged nucleic acid molecule (for example, DNA) entering target cells, and used in gene delivery.

Exemplary NBs and MBs according to the present invention have structure shown in FIG. 1. The SF₆ (or equivalent) gas filled in the NBs and MBs of this invention may exhibit US imaging functionality. In some embodiments of the present invention, the bubble shell is made of an amphiphilic chitosan, and the hydrophobic agent and lipophilic superparamagnetic iron oxide (SPIO) nanoparticles are encapsulated in the bubble shell for functioning as MR contrast agent. The bubble is bombarded under a medical diagnostic ultrasound of middle-low frequency (20-100 k Hz) or high frequency (1-12 MHz), and ordinary power density (2.4 W/cm² or less), thus exhibit a US-triggered drug releasing function.

After the structure of nano-/micro-bubble shell is destroyed by the ultrasound bombardment, the amphiphilic chitosan molecules in the bubble shell will be dispersed with carrying the hydrophobic agent, and the dispersed amphiphilic chitosan molecules will further increase solubility of the hydrophobic agent in aqueous physiological environment, which will help the dispersed hydrophobic agent to be internalized by target cells for more easier to reach the cells. Moreover, the amphiphilic chitosan molecules will enhance the opening of cell membrane, and improve the transmembrane delivery of hydrophobic agent into target cell with the synergistic effect on temporary cell membrane opening by the seismic wave after ultrasound bombardment.

In another embodiment of the present invention, an amphiphilic chitosan, lipophilic fluorescent nanoparticles (for example, zinc oxide or quantum dot nanoparticles), and a hydrophobic anti-cancer agent are used to form a US-triggered drug releasing nano-/micro-bubble exhibiting encapsulation of a hydrophobic drug and optical imaging functionalities by hydrophobic interactions. The hydrophobic agent and lipophilic fluorescent nanoparticles are encapsulated in the bubble shell for functioning as optical contrast agent, by which the internalization of nano-/micro-bubbles and drug release triggered by US can be observed under a confocal microscope.

In one embodiment of the present invention, said bubble is composed of amphiphilic chitosan, and contains SF₆ or other equivalent gas, such as C₃F₈. As required, size of the microbubbles may be controlled in the range of 0.7 μm to 5 μm through the manufacturing process technology. The nano-/micro-bubble of this invention may create US imaging effects by itself, or may release hydrophobic agent after the bombardment performed by a US system operated under an appropriative mechanical index (MI).

In one embodiment of the present invention, said nano-/micro-bubble shell is composed of amphiphilic chitosan, and contains an internal gaseous core of SF₆ or other equivalent gas. The hydrophobic group on the amphiphilic chitosan structure will cause binding of lipophilic imaging particles and the hydrophobic agent onto the inner surface faced to the gaseous core by hydrophobic interactions, and thus provide capacity and a space for carrying the hydrophobic agent.

In the case without US bombardment, hydrophobic interaction between the lipophilic imaging nanoparticles and the amphiphilic chitosan will function as a physical cross-linkage to stabilize bubble structure and prevent natural explosion. Besides, the lipophilic imaging nanoparticles may function as stabilizer for limiting the boundary of bubble, which may inhibit over-growing of bubbles (as shown in FIG. 2). The lipophilic particles also prevent leakage of drugs in the absence of ultrasound sonication, which can result in a significant release of drug after the explosion of bubbles under the ultrasound bombardment.

In another aspect, this invention provides a hydrophobic drug delivery system, which is characterized by comprising the nano-/micro-bubble of present invention. In hydrophobic drug delivery system, the location and concentration of drug vehicle can be monitored through imaging technique, and drug release from vehicle can be triggered by a medical diagnostic ultrasound, which may effectively improve the precision of administration timing at target site, and reduce the toxicity of chemotherapeutic drugs to normal tissues.

In one embodiment, the nano-/micro-bubble of present invention uses lipophilic superparamagnetic nanoparticles as the imaging particles, which may function as an MR T2 contrast agent in the drug delivery system before US triggering. The dispersive extent of superparamagnetic nanoparticles will be changed after the action of ultrasound triggering, there will be differences between T2 and T2* contrast signals before and after the action of ultrasound, and can be used as references for judging the state of vehicles (where it is triggered to break and release drugs).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic drawing showing the structure and action principle of one embodiment of the nano-/micro-bubble with ultrasound-triggered release and imaging functionalities of present invention.

FIG. 2 is a schematic drawing showing the formation principle of one embodiment of the nano-bubble with ultrasound-triggered release and imaging functionalities of present invention.

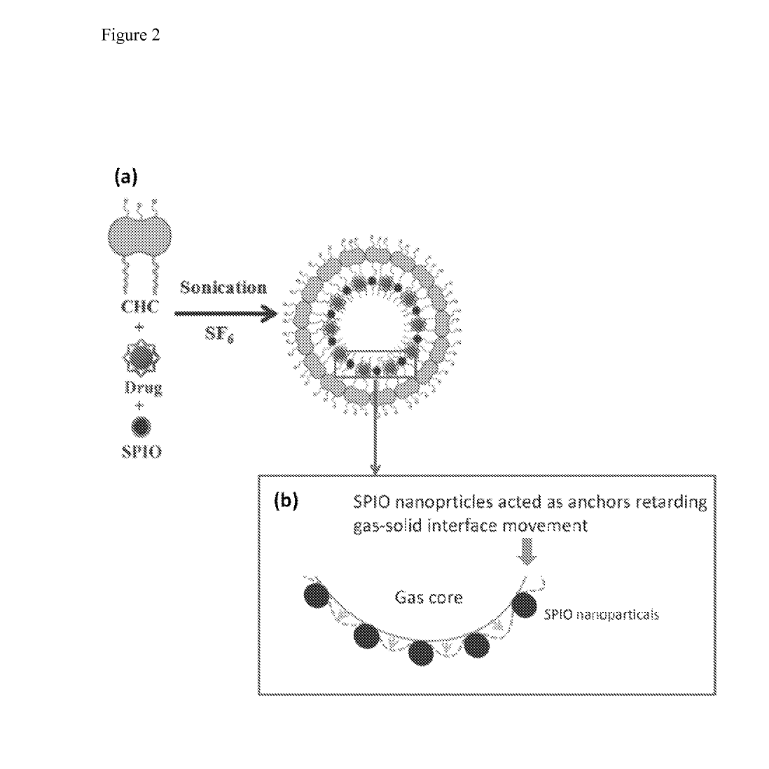

FIG. 3 is a diagram showing TEM photographs of CHC MB (FIG. 3(a)), CHC/SPIO MB (FIG. 3(b)), and CHC/SPIO NB (FIG. 3(c)) prepared in one embodiment of the present invention. FIG. 3(d) shows HR-TEM photograph of the SPIO nanoparticles incorporated in the shell of CHC/SPIO NB. FIG. 3(e) shows photographs of the CPT-loaded CHC/SPIO NB suspension with magnetism under visible and ultraviolet (UV) light irradiation. FIG. 3(f) shows the Magnetization-magnetic field strength curve of CHC/SPIO NBs.

FIG. 4(a) is a diagram showing the particle size analysis of CHC MB, CHC/SPIO MB, and CHC/SPIO NB prepared in one embodiment of the present invention. FIG. 4(b) and FIG. 4(c) show the results of optical microscope observation of CHC MB and CHC/SPIO MB suspension maintained at 4° C. for 7 days, respectively. Scale bar 20 μm.

FIG. 5 is a diagram showing the confocal microscope observation for CPT-loaded CHC/SPIO NBs (FIG. 5(a)) and FITC-loaded CHC/SPIO NBs (FIG. 5(b), indicated by arrows). FIG. 5(c) shows the effect of SPIO amount on the encapsulation efficiency of CHC/SPIO bubbles. FIGS. 5(d) and (e) show the confocal microscope observation for CPT-loaded CHC/SPIO NBs after the release test without sonication and with sonication (ultrasonic frequency: 1 MHz, power density: 1 W/cm², duty ratio: 20%) for 20 min, respectively. FIG. 5(f) shows the confocal microscope observation for CPT-containing medium (without CHC/SPIO NBs) under sonication (ultrasonic frequency: 1 MHz, power density: 1 W/cm², duty ratio: 20%) for 20 min. Scale bar 5 μm.

FIG. 6 is a diagram showing the cell viability of breast tumor cells (MDA-MB-231) cultured under different conditions. In FIG. 6(a), the control group was performed on the cells cultured without adding the model drug (CPT) and performing sonication. In FIG. 6(b), cells were sonicated for 20 min. In FIG. 6(c), cells were cultured with the medium containing 75 μL of CPT-loaded CHC/SPIO NB suspension without sonication for 20 min. In FIG. 6(d), cells were cultured with the medium containing 75 μL of CPT-loaded CHC/SPIO NB suspension with sonication for 20 min. In FIG. 6(e), cells were cultured with CPT-containing medium (CPT/medium concentration: 1.3 μg/mL) under sonication for 20 min. Dash line indicates that the cell viability of breast tumor cells cultured with CPT-containing medium at a concentration of 1.3 μg/mL without sonication for 20 min. After above treatments, the cell mediums were removed and the cells were washed with PBS. Then, fresh medium was added and further cultured for 24 hr. The cell viability was measured by MTT assay.

FIG. 7 is a diagram showing the confocal microscope observation of MDA-MB-231 cells cultured with different samples. In FIG. 7(a), the control group was performed on the cells cultured without adding the model drug (CPT) and performing sonication. In FIG. 7(b), cells were sonicated for 20 min. In FIG. 7(c), cells were cultured with the medium containing 75 μL of CPT-loaded CHC/SPIO NB suspension without sonication for 20 min. In FIG. 7(d), cells were cultured with the medium containing 75 μL of CPT-loaded CHC/SPIO NB suspension with sonication for 20 min. In FIG. 7(e), cells were cultured with CPT-containing medium (CPT/medium concentration: 1.3 μg/mL) under sonication for 20 min. After above treatments, the cell mediums were removed and the cells were washed with PBS. Subsequently, fresh medium was added to all culture dishes for a confocal microscopic observation. Scale bar 20 μm.

FIG. 8 is a diagram showing the in vitro and in vivo ultrasonic images of samples. FIG. 8(a) and FIG. 8(b) show the in vitro US images of CHC MB (9.3×10⁷ bubbles/mL) and CHC MB (9.3×10⁷ bubbles/mL), respectively. FIGS. 8(c) and 8(d) show the in vivo US images of commercialized MB (Sono Vue MB) suspension (1.8×10⁸ bubbles/mL) acquired at a vein before and after injection, respectively. FIGS. 8(e) and 8(f) show the in vivo US images of CHC/SPIO MB suspension (1.8×10⁸ bubbles/mL) acquired at a vein before and after injection, respectively.

FIG. 9 is a diagram showing the in vitro MR images of mouse liver acquired before and after injection of CHC/SPIO NB suspension.

### DETAILED DESCRIPTION OF THE INVENTION

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Further, any mechanism proposed below does not in any way restrict the scope of the claimed invention.

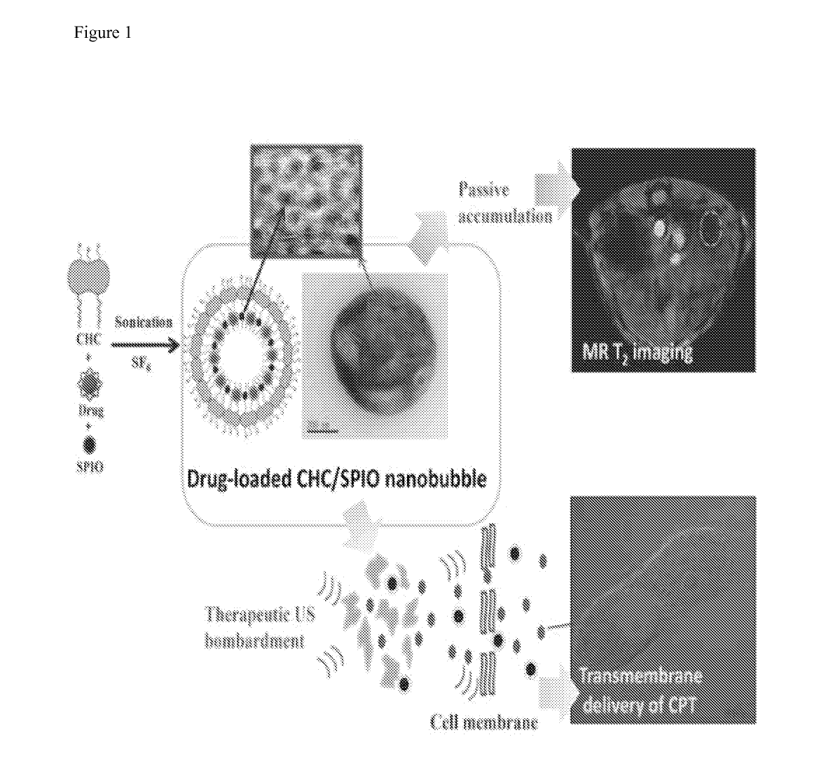

The main objective of the present invention is to use amphiphilic chitosan as a new bubble-forming material, combined with lipophilic superparamagnetic iron oxide (SPIO) nanoparticles to prepare a nano-/micro-bubble (CHC/SPIO NBs and CHC/SPIO MBs) with encapsulation of hydrophobic agent, US-triggered release, and MR imaging functionalities; or combined with lipophilic zinc oxide or quantum dot nanoparticles to prepare a nano-/micro-bubble with encapsulation of hydrophobic agent, US-triggered release, and optical image contracting functionalities. Therefore, the following description describes the CHC/SPIO NBs and CHC/SPIO MBs as preferable examples of this invention. As shown in FIG. 1, the multifunctional MR image-guided drug delivery vehicle comprises a micro- or nano-bubble containing a water-insoluble gas (for example, SF₆ or other equivalent gas, such as C₃F₈). The shell of said bubble is composed of amphiphilic chitosan and lipophilic superparamagnetic iron oxide (SPIO) nanoparticles, and a hydrophobic anti-cancer agent is encapsulated inside the bubble shell.

As a preferable embodiment of this invention, a CHC/SPIO nano-bubble is prepared as follow. NOCC (N,O-carboxymethyl chitosan) (2 g) were dissolved in distilled (DI) water (50 mL) and stirred for 24 h. The resulting solutions were mixed with methanol (50 mL), and further stirred for 24 h. After through mixing of the solution, hexanoic anhydride was added at concentrations of 0.5 M. After complete reaction and drying processes of the product at 50° C. for one day, an amphiphilic chitosan derivative CHC (carboxymethyl hexanoyl chitosan) was obtained.

The mixture of 5 mL CHC aqueous solution (1.5% w/w), 0.05 g of glucose (3.3 mg/mL), and 10 mL distilled (DI) water was purged with SF₆ gas, and sonicated using a probe-type sonicator (XL2000, Misonix Inc., USA) for 3 min. A predetermined amount (20 μL, 50 μL or 100 μL) of superparamagnetic iron oxide suspension in ethanol, and 65 μL of hydrophobic agent (camptothecin, CPT) solution (CPT/ethanol solution, 12 mg/ml) were added, and sonicated with a US homogenizer. After sonication, the CPT-loaded CHC/SPIO bubbles with different particle size were obtained as a milky suspension.

The shell of nano-/micro-bubble of present invention is mainly made of amphiphilic chitosan. The hydrophilic group will distribute outwardly when the bubble is formed, which made the bubble surface hydrophilic and maintain the dispersity of whole structure in aqueous solution. Furthermore, the bubble surface contains NH₂ group and COOH group, thus the nano-/micro-bubble is charged in a physiological environment. The surface potential can be controlled by adjusting the grafting ratio of carboxymethyl and hydrophobic groups in the synthetic process of amphiphilic chitosan. Therefore, the surface potential of bubbles may be adjusted according to different physiological requirements, such as blood compatibility and circulating time of the drug vehicle in body, for a broader adaptability to biomedical application. The NH₂ and COOH groups on the bubble surface may easily conjugate to a ligand by peptide bonding or other interactions, which can perform a more efficient targeted delivery.

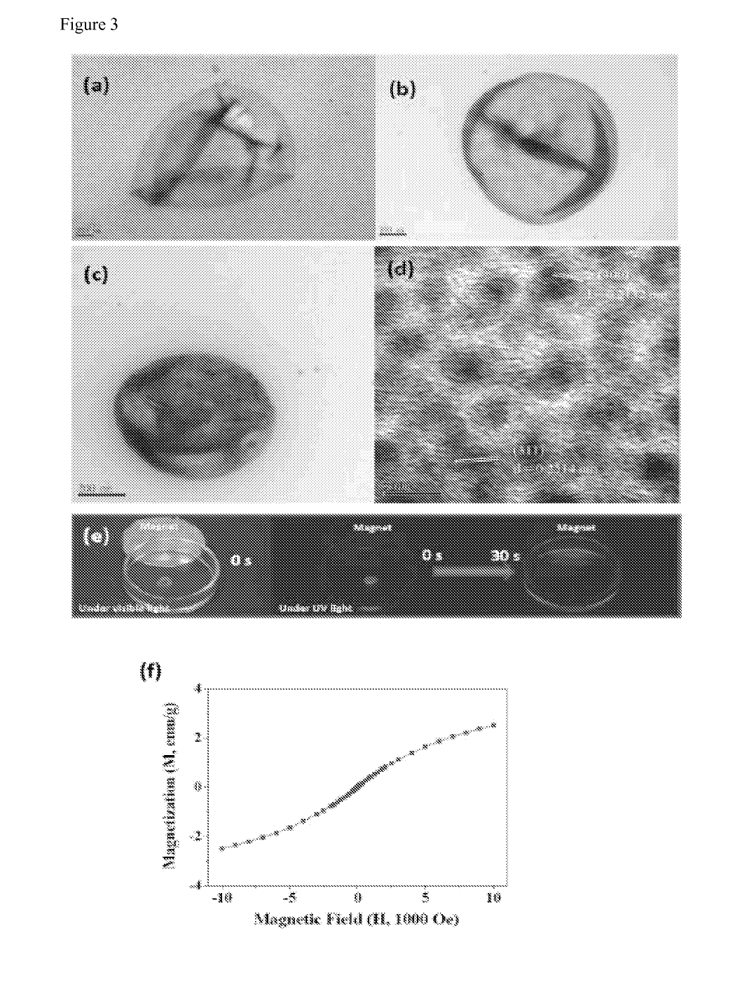

FIG. 3 shows TEM photographs of CHC MB, CHC/SPIO MB, and CHC/SPIO NB prepared in one embodiment of the present invention using CHC as a bubble-forming material. As shown in FIGS. 3(a) and (b), CHC MB and CHC/SPIO MB both demonstrated a hollow structure suggesting that CHC exhibited excellent bubble-forming ability for preparing MBs by a facile sonication method. In addition, it was found that the shell matrices of CHC/SPIO NBs was darker than that of CHC MBs owing to the incorporation of nanoparticles, suggesting that the incorporation of SPIO could efficiently decrease the size of CHC microbubbles. Thus, it is possible to prepare CHC NBs by adjusting the amount ratio of the inorganic nanoparticle. The phase of the nanoparticles incorporated in the shell of CHC-based bubbles was characterized by high-resolution (HR) TEM. As shown in FIG. 3(d), SPIO nanoparticles were evenly dispersed in the bubble shell, suggesting the magnetic property of CHC NBs. FIG. 3(e) shows a photograph of CPT-loaded CHC/SPIO NBs with magnetic attraction; it is observed that CPT-loaded CHC/SPIO NBs demonstrating blue luminescence were attracted by the magnet. This result also demonstrated magnetism of the bubbles prepared in the present invention. The magnetic properties of the CHC/SPIO NBs were also characterized using a SQUID. FIG. 3(f) shows their magnetization-magnetic field strength (M-H) curve; no hysteresis loop was observed, implying that the proposed vehicle exhibited superparamagnetic behavior that was expected to demonstrate MR T2 image contrast.

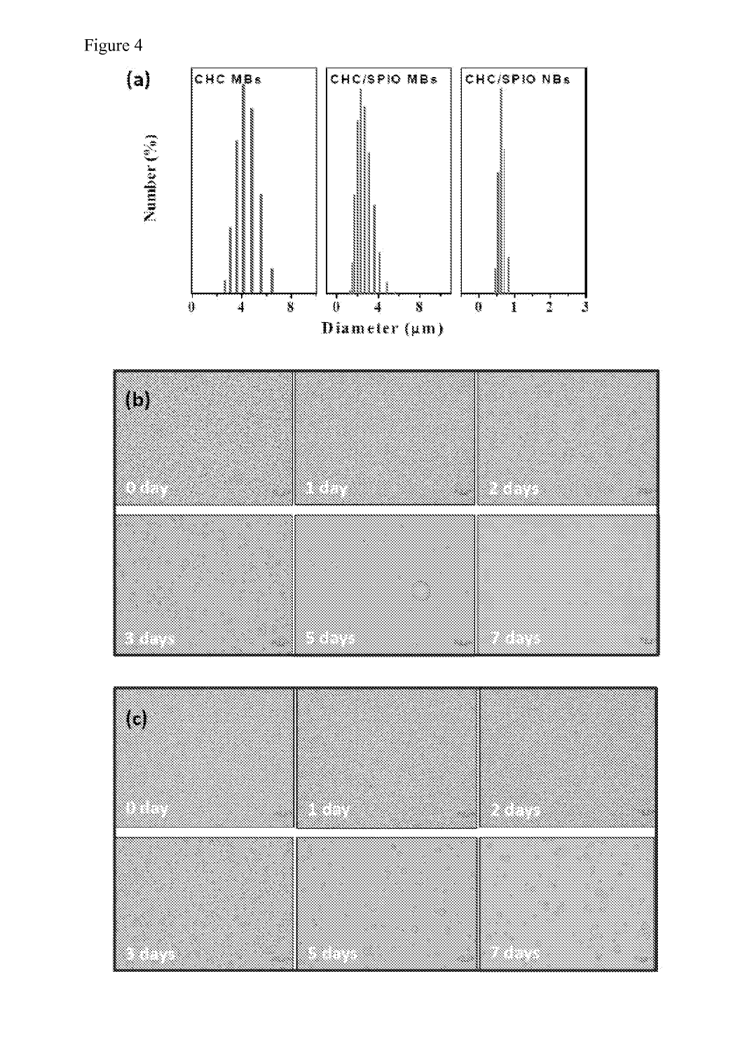

FIG. 4(a) shows the particle size distribution of CHC MBs, CHC/SPIO MBs, and CHC/SPIO NBs. The results of particle size analysis suggested that CHC may be used as bubble-forming material for preparing MBs and NBs. As shown in FIGS. 4(b) and 4(c), both CHC MBs and CHC/SPIO bubbles tend to spontaneously merge together, continuously grow up, and disrupt owing to the thermodynamic driving force (i.e., the overall surface energy of the bubbles tends to a minimal value). It can be observed that the stability of CHC/SPIO MBs was much higher than that of CHC MBs. This was probably attributable to the lipophilic SPIO nanoparticles that exerted a physical cross-linking effect to crosslink the hydrophobic moieties of CHC molecules.

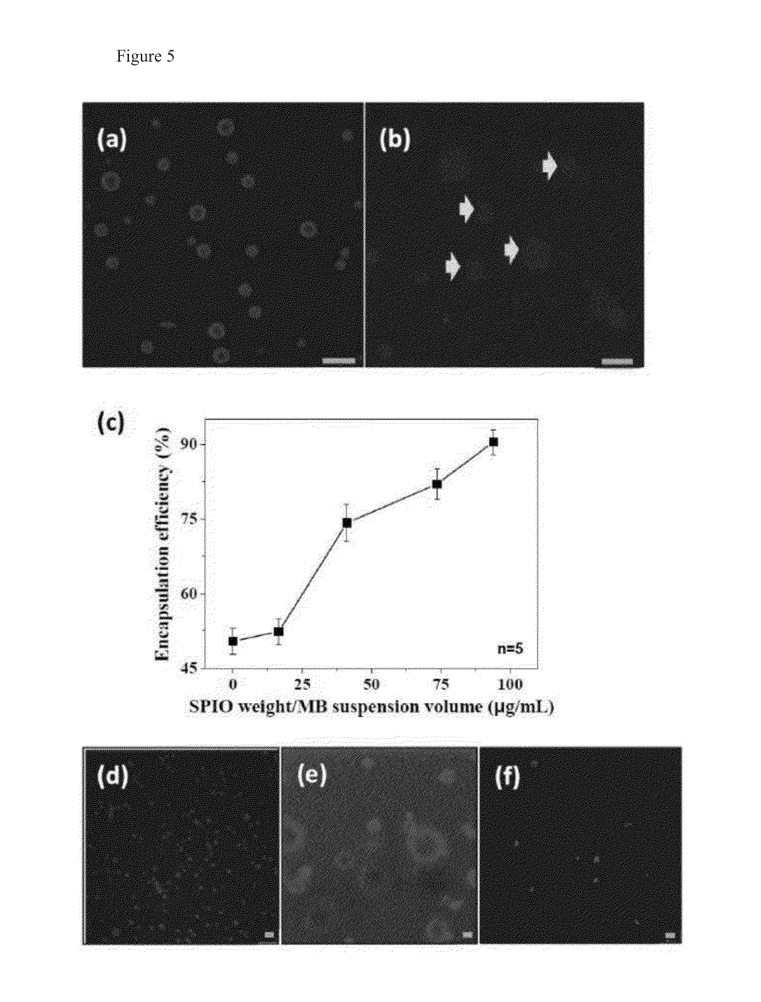

The confocal photographs of the as-received CHC/SPIO MBs loaded with hydrophobic agent (CPT, blue fluorescence) and hydrophilic agent (FITC, green fluorescence) are shown in FIGS. 5(a) and 5(b), respectively. As shown, the CPT-loaded CHC/SPIO MBs demonstrated significant blue fluorescence [FIG. 5], but FITC-loaded CHC/SPIO MBs demonstrated only very weak green fluorescence [FIG. 5(b)]. This implies that CHC/SPIO MBs can carry both hydrophobic and hydrophilic agents, and the encapsulation ability of CHC/SPIO MBs for CPT (a hydrophobic agent) was better than that for FITC (a hydrophilic agent). In FIG. 5(c), it was found that the encapsulation efficiency of the hydrophobic model drug, CPT, increased with the SPIO content. This suggests that lipophilic SPIO nanoparticles facilitated the retention of more hydrophobic drug inside the bubbles by providing a hydrophobic interaction and diffusion barrier. As shown in FIGS. 5(d) and 5(e), CHC/SPIO NBs slightly grew up and exhibited a low background leakage for encapsulating hydrophobic agents after sonication. Hydrophobic CPT was well-dispersed in hydrophilic medium because the amphiphilic CHC molecules, from disrupted CHC/SPIO bubbles, acted as a biocompatible surfactant to emulsify CPT. This was not observed for the CPT-containing medium (without CHC) under the same sonication for 20 min. As shown in FIG. 5(f), CPT was not well-dispersed under sonication because the absence of CHC. These results suggested that the synergistic effect of CHC and sonication may increase solubility and dispersion of hydrophobic agent in aqueous solution.

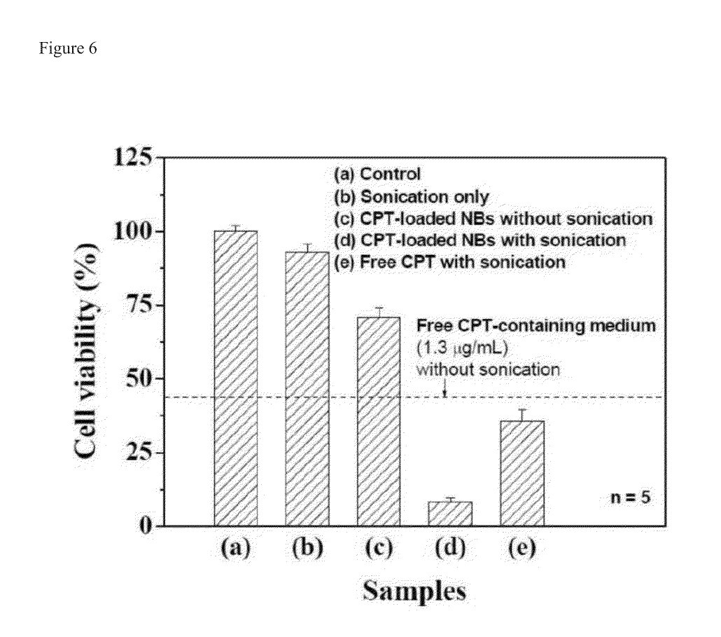

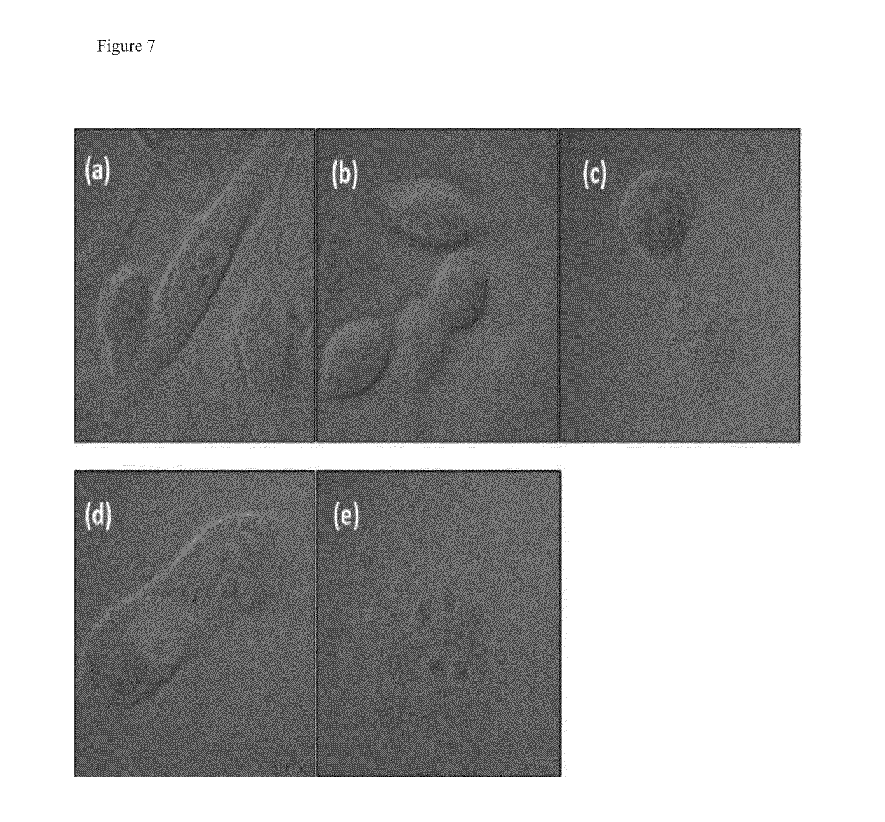

FIG. 6 shows the effect of US triggering on the in vitro cytotoxicity of the CPT-loaded CHC/SPIO NBs to tumor cells. It was found that the cell viability of tumor cells sonicated with safe parameters [column (b)] was not significantly different from that of the control group [columns (a)]. In comparison, free CPT-induced cytotoxicity (dash line) could be lowered by encapsulating CPT in CHC/SPIO NBs [column (c)], and the CPT-loaded CHC/SPIO NBs demonstrated a high tumor cell killing ability under sonication [column (d)]. In FIG. 6(e), cancer cells those contacted free CPT (non-encapsulated by CHC/SPIO NB) and subjected to sonication exhibited higher viability than those treated with CPT-loaded CHC/SPIO NBs under sonication. Thus, CPT molecules could efficiently access tumor cells. Moreover, the amphiphilic CHC molecules from ruptured bubbles also might be helpful to increase transmembrane permeation. This can be supported by the microscopic observation of cells immediately taken after release treatments, as shown in FIG. 7.

FIG. 7 is a diagram showing the confocal microscope observation of cancer cells treated with the bubbles prepared in according to one embodiment of present invention (CPT-loaded CHC/SPIO NBs) with or without sonication. A comparison of FIG. 7(d) with FIGS. 7(a)-7(c) and 7(e) shows that after sonication, it was found that green fluorescent CPT observed in the tumor cells cultured with CPT-loaded CHC/SPIO NBs under sonication [FIG. 7(d)] was more significant than that in the tumor cells cultured with free CPT under sonication [FIG. 7(e)]. In addition, it was found that the green fluorescence assigned to the CPT uptake was not clearly observed in FIG. 7(c). This was ascribed to the enhanced drug dispersion and transmembrane delivery resulted from the synergistic effect of surfactant-like CHC molecules and sonication.

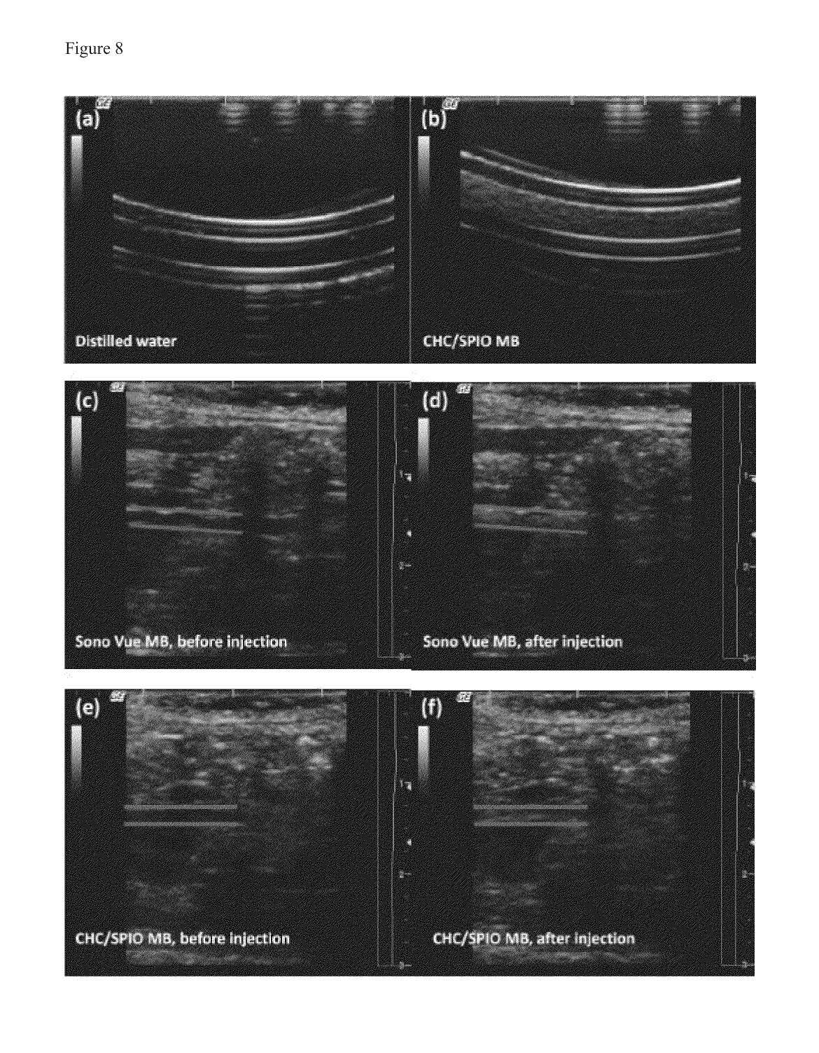

FIG. 8 shows the comparative results of CHC/SPIO MBs prepared in according to one embodiment of present invention with a commercial US contrast product (Sono Vue MBs) in imaging ability. FIG. 8(c) and FIG. 8(d) show the vascular image before and after delivering the commercial product (Sono Vue MBs), respectively. FIG. 8(e) and FIG. 8(f) show the vascular image before and after delivering CHC/SPIO MBs, respectively. As shown in FIG. 8(f), CHC/SPIO MBs are clearly visible in a vein in a manner similar to the vascular image of Sono Vue [FIG. 8(d)]. It is suggested that CHC is a potential candidate material for preparing MBs as a US contrast agent.

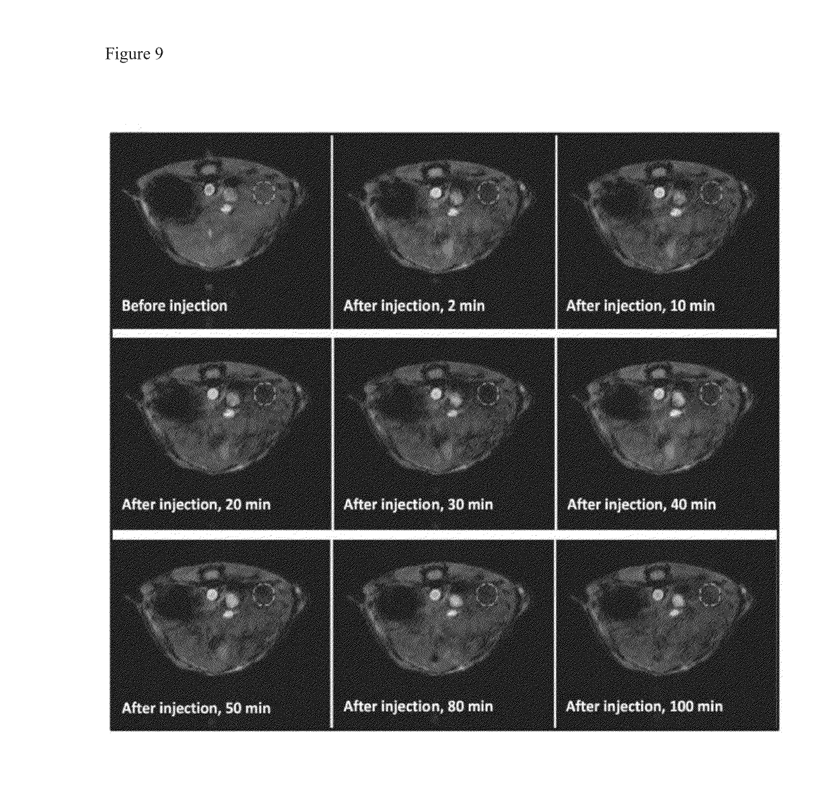

As shown in FIG. 9, we focused on sliced MR images of the liver to evaluate the MR T2 imaging capacity of CHC/SPIO microbubbles of this invention. For CHC/SPIO NBs, the T2 contrast of the MR image was promptly enhanced in the liver in the first 2 min after injection, suggesting that the CHC/SPIO MBs can be employed as a candidate material for MR-image-guided applications. Subsequently, the in vivo MR imaging ability was characterized by using CHC/SPIO NBs. On the other hand, both the CHC/SPIO NBs and MBs demonstrated significant MR T2 imaging contrast.

In conclusion of the experimental results described above, the novel drug-loaded NBs and MBs with imaging and US-triggered release functionalities prepared by the present invention still maintain their US and MR imaging property when administered to an animal body, and can enter circulating system of animal body as a drug delivery vehicle. In summary, the ultrasound triggered drug delivery system of the invention combines MRI tracking of vehicles with in vitro ultrasound triggered drug release by using medical diagnosis ultrasound, which can effectively improve the precision of administration position and timing at target site, and reduce the toxicity of chemotherapeutic drugs to normal tissues.

Also, the drug releasing methods of this invention uses ultrasound as the triggering energy, is enable to trigger vehicle breaking and drug release by using medical diagnosis ultrasound, which has been highly commercialized. The medical ultrasound with high security also has advantages of energy focusing, precise directional transmission, deeply penetrating soft tissue and the like. Furthermore, ultrasound has the function of accelerating drug penetration and absorption, which has been widely used in clinical treatment for transdermal administration, cancer treatment and physical therapy.

In addition, the medical fields have clinical experience in integrating the diagnostic ultrasound into magnetic resonance imaging equipments. Therefore, the technology described in the present invention can be practiced by using existing equipments, and is an invention possessing both practicability and safety.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A ultrasound (US)-triggered drug releasing nano-/micro-bubble exhibiting encapsulation of a hydrophobic drug and imaging functionalities, which comprises:
a bubble shell composed of an amphiphilic chitosan material;
a gaseous core filled of a water-insoluble gas; and
a hydrophobic agent dispersed in the shell incorporated with hydrophobic nanoparticles by a hydrophobic interaction.

2. The US-triggered drug releasing nano-/micro-bubble of claim 1, which further comprises a lipophlic imaging nanoparticle.

3. The US-triggered drug releasing nano-/micro-bubble of claim 1, wherein the nano-bubble has a particle size of 600-900 nm.

4. The US-triggered drug releasing nano-/micro-bubble of claim 1, wherein the micro-bubble has a particle size of 3 μm to 10 μm.

5. The US-triggered drug releasing nano-/micro-bubble of claim 2, wherein the lipophlic imaging nanoparticle is distributed inside the bubble shell.

6. The US-triggered drug releasing nano-/micro-bubble of claim 1, wherein the amphiphilic chitosan material is carboxymethyl hexanoyl chitosan (CHC).

7. The US-triggered drug releasing nano-/micro-bubble of claim 2, wherein the lipophlic imaging nanoparticle has a particle size of 1 nm to 20 nm.

8. The US-triggered drug releasing nano-/micro-bubble of claim 1, wherein the hydrophobic agent is a hydrophobic anti-cancer agent.

9. The US-triggered drug releasing nano-/micro-bubble of claim 1, wherein the hydrophobic agent is a negatively charged nucleic acid molecule.

10. The US-triggered drug releasing nano-/micro-bubble of claim 1, wherein the water-insoluble gas includes SF₆ or C₃F₈.

11. The US-triggered drug releasing nano-/micro-bubble of claim 2, wherein the lipophlic imaging nanoparticle is a lipophilic superparamagnetic iron oxide (SPIO) nanoparticle.

12. The US-triggered drug releasing nano-/micro-bubble of claim 2, wherein the lipophlic imaging nanoparticle is an optical imaging nanoparticle.

13. The US-triggered drug releasing nano-/micro-bubble of claim 12, wherein the optical imaging nanoparticle includes a zinc oxide or quantum dot nanoparticle.

14. The US-triggered drug releasing nano-/micro-bubble of claim 1, wherein the amphiphilic chitosan material is an amphiphilic chitosan having hydrophilic carboxymethyl groups and lipophilic acyl groups.

15. The US-triggered drug releasing nano-/micro-bubble of claim 1, which is characterized by the nano-/micro-bubble being broken under the ultrasound bombardment of middle-low frequency (20-100 k Hz) or high frequency (1-12 MHz) and low power density (below 3 W/cm²).

16. A drug delivery system for hydrophobic agent, which is characterized by comprising the nano-/micro-bubble of claim 2 and a pharmaceutically acceptable diluent, vehicle or excipient.

17. A drug delivery method by ultrasonically-triggered vehicle, which comprises the step of:
administrating the drug delivery system for hydrophobic agent of claim 16 to a subject in need thereof;
tracking the image of drug vehicle through medical imaging; and
triggering drug release by ultrasound (as triggering energy) when the vehicle reaching to target site and accumulating to effective amount of treatment.

18. The method of claim 17, wherein the vehicle is bombarded by ultrasound of middle-high frequency and low power density, which results in destroying the drug vehicle structure and no longer assembling (or re-coating) to release the drug.

19. The method of claim 17, wherein the synergistic effect created by the amphiphilic chitosan material constructing the nano-/micro-bubble shell and ultrasonication accelerates the transmembrane delivery of the hydrophobic agent into target cell, and enhances the toxicity of the agent to the cell.

20. The method of claim 17, which further comprises the step of detecting drug releasing state of the vehicle through the alteration in magnetic resonance (MR) signal caused by the structural change of the drug vehicle.

21. The method of claim 20, wherein the MR signal variation refers to the significant change in the difference value between R2* slope and R2 slope.
